(19) United States
(12) Patent Application Publication
(10) Pub. No.: US 2011/0040204 A1
(43) Pub. Date: Feb. 17, 2011

(54) NEUROLOGICAL STIMULATION AND ANALYSIS

(75) Inventors: Antoni Ivorra, Berkeley, CA (US); Boris Rubinsky, Givataaim (IL)

Correspondence Address: UC Berkeley - OTL;Bozicevic, Field & Francis LLP, 1900 University Avenue, Suite 200, East Palo Alto, CA, 94303, US

(73) Assignee: The Regents of The University of California, Oakland, CA (US)

(21) Appl. No.: 12/445,734

(22) PCT Filed: Oct. 19, 2007
(86) PCT No.: PCT/US07/22304

§ 371 Date: Sep. 30, 2010

Related U.S. Application Data
(60) Provisional application No. 60/852,746, filed on Oct. 19, 2006.

(51) Int. Cl. A61B 5/16; A61B 5/11

(52) U.S. Cl. 600/557; 600/595

(57) ABSTRACT

In one embodiment, a method for neurological interrogation and response analysis is provided. The method includes inducing, at the device, a non-optical excitation for a user. The device comprises a wearable device being worn by the user such that the non-optical excitation can be sensed. A response of a voluntary conscious movement is detected from the user. An outcome is analyzed based on the response and the non-optical excitation. An action can be performed in response to the analysis.

### CROSS REFERENCES TO RELATED APPLICATIONS

This application claims priority from U.S. Provisional Patent Application Ser. No. 60/852,746, entitled “NEUROLOGICAL STIMULATION AND ANALYSIS”, filed on Oct. 19, 2006, which is hereby incorporated by reference as if set forth in full in this application for all purposes.

### BACKGROUND

Particular embodiments generally relate to a neurological interactive stimulation, interrogation, and analysis device.

Current automated interactive systems are based on computerized tests that are much too cumbersome or obtrusive to allow continuous monitoring. That is, those systems would interfere with normal life activities. For example, the user needs to sit at a desk and interact with a desktop computer to perform the test. The same occurs with rehabilitation systems based on the generation of artificial stimuli to which the patient must respond.

Currently, neurological assessment is performed with test batteries that can be automated by the use of computer devices. Following current trends of computer miniaturization and pervasiveness, those tests have been embedded in smaller computers that can be used at home. However, even in the case of computers, such as advanced mobile phones, the user must stop whatever activity he or she is doing in order to watch a display and to press buttons to perform the test. These tests require the use of vision in test subjects capable of vision. At the end, this constrains the examination and also implies that truly continuous assessment is not possible.

### SUMMARY

In one embodiment, a method for neurological interrogation and response analysis is provided. The method includes inducing, at the device, a non-optical excitation for a user. The device comprises a wearable device being worn by the user such that the non-optical excitation can be sensed. A response of a voluntary conscious movement is detected from the user. An outcome is analyzed based on the response and the non-optical excitation. An action can be performed in response to the analysis.

A further understanding of the nature and the advantages of particular embodiments disclosed herein may be realized by reference of the remaining portions of the specification and the attached drawings.

### BRIEF DESCRIPTION OF THE DRAWINGS

FIG. 1 shows an example of a neurological assessment device.

FIG. 2 depicts a simplified flowchart of a method for performing a neurological test according to one embodiment.

FIG. 3 shows an example of the peripheral nervous system and central nervous system according to one embodiment.

FIG. 4 shows an example of the device according to embodiments of the present invention.

FIG. 5 shows another example of the device according to one embodiment.

FIG. 6 shows yet another example of a neurological assessment device according to one embodiment of the present invention.

FIG. 7 shows an example of a “Reaction time test.”

FIG. 8 shows an example of a “Pace rate estimation.” test.

FIG. 9 depicts a flowchart showing a method according to one embodiment of the present invention.

FIG. 10 shows an embodiment of a neurological assessment device.

### DETAILED DESCRIPTION OF EMBODIMENTS

There are different situations in which it would be valuable to use systems to continuously assess or excite the neurological capabilities of individuals (e.g., diabetic patients under hypoglycemic risk, Alzheimer's disease patients, control of delivery of neural drugs or treatments, neuro-toxicology studies and persons performing potentially dangerous tasks such as car driving or plane piloting, paralyzed patients in rehabilitation). In those cases, the results could be employed, for instance, to trigger alarms, to monitor patient evolution under treatment, to execute therapeutic maneuvers, to disable certain mechanisms or for legal purposes. In addition, for rehabilitation purposes, a wearable device able to generate stimuli to which patient must respond in a continuous fashion is useful. For example, when a limb is paralyzed from an accident and there is need for training to recover the limb functionality, such device could be employed to remind occasionally the patient to try to move the limb or to provide intelligent-feedback control stimulation sequences that would optimize the recovery of the limb.

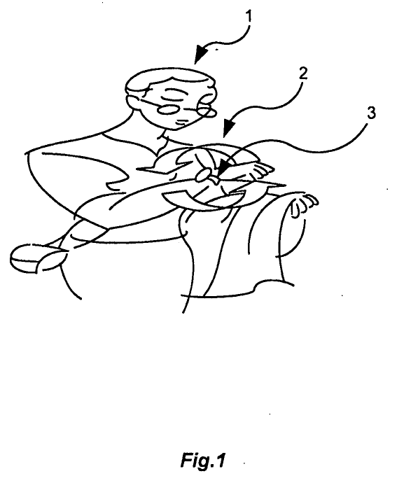

FIG. 1 shows an example of a neurological interrogation and assessment device. Device 3 may be integrated into a watch or other wearable device, such as a watch, gloves, arm, leg or body bands, hearing aids type device, Bluetooth device, glasses, or goggles. Particular embodiments not only serve as a platform for solutions requiring non-invasive mental assessments, but can also be integrated into other related systems. For example, a device for measuring neural ability may be integrated into products that already exist such as radio-frequency panic buttons, GPS location devices, actimeters and schedulers for medication. Also, the device may be embedded in an implantable capsule.

Device 3 may formulate non-obtrusive neurological tests that minimize the impact on the user's activity. In one embodiment two kinds of neurological tests can be used:
Non-Distracting Tests” (NDT)—Questions asked through non-optical excitations (e.g., acoustic, vibration, smell, heat or cold or electrical stimulation) and answers are provided by subtle user movements. The NDTs may or may not be preceded by a warning signal.
Attention Demanding Tests” (ADT)—These tests may be tests in which various sequences of signals of greater complexity than in the NDT are produced by the device. Thus, the user may interrupt whatever activity he is performing in order to answer a test. For example, the ADT may require a user to use optical means to answer a question. The attention demanding tests can be used, for instance, to confirm a low-score in a NDT before triggering any event such as an alarm. In contrast to ADT, particular embodiments allow a user to use simple hand movements that require no optical intervention to respond rather than having to press buttons or use a tactile screen. It will be understood that optical tests may be added to the NDT at some point.

The minimally intrusiveness of the tests is achieved by using non-optical interrogations provided by a user-machine interface. Non-optical interrogations may be interrogations that do not require a user to use optical senses to sense the interrogation and/or respond to the interrogation using optical methods. Examples of non-optical interrogations include acoustic signals (by means of a speaker or buzzer), excitations of the sense of smell or, haptic excitation signals vibration (by means of piezoelectric actuator or an electromagnetic actuator), local pressure or non-painful electrical stimulation (by means of electrodes), or local heating or cooling. The user can then respond to the interrogations. For example, referring to FIG. 1, the user 1 responds by subtle wrist movements 2 in a defined pattern, such as a counterclockwise or clockwise turn of the wrist. Also, non-voluntary responses may also be detected. These may be reflex actions, changes in temperature, etc. The response can be detected by device 3 by a response detector, such as embedded movement sensors (e.g., accelerometers, gyroscopes or inertial switches). The results, such as measured response time and accuracy, are stored in memory or sent externally through a wired or wireless link. In another embodiment, device 3 compares the results with pre-stored thresholds and decides whether or not to trigger an alarm. These thresholds may be determined from a baseline neurological assessment for each user and may be modified by different parameters such as time (to take into account circadian rhythms).

Device 3 contains components to generate non-optical signals that are employed to ask the user to perform some kind of neurological exercise. The user will notice these signals. For example, the user may be wearing the device or have the device in his/her pocket. By wearing the device, the device is placed in a position such that the user can sense the signals. For example, the user can feel the vibrations or hear the audible sounds. The device also contains sensors to detect specific user movements that do not require visual response. The user may use those movements to respond to the neurological exercises.

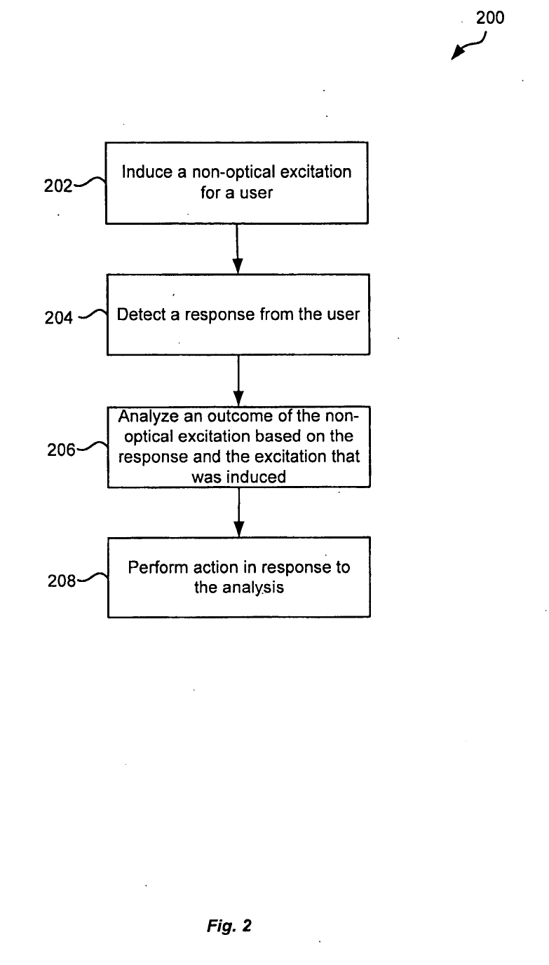

FIG. 2 depicts a simplified flowchart 200 of a method for performing a neurological test according to one embodiment. In step 202, device 3 induces a non-optical excitation for a user. A non-optical excitation may be an interrogation posed that does not require optical methods to detect the question and/or respond to the question. For example, the user may be wearing device 3 such that the non-optical excitation can be sensed. By wearing, the device does not have to be touching the user. Rather, the device is positioned such that the non-optical excitation can be sensed. For example, the non-optical excitation may be a vibration, change in temperature, an electrical current, etc. that can be sensed by the user.

In step 204, device 3 detects a response from the user. The response may be a non-intrusive response. For example, the response may not require a visual or optical action to be taken by the user, such as the user may perform a movement that device 3 can detect. The movement may be in a pre-defined pattern and/or speed/acceleration to indicate the response. This may be different from a user having to look at device 3 and select a button or perform any other optical alignment that may be needed to provide the response. For example, the user does not have to align his/her fingers with a keyboard to select the response on the keyboard.

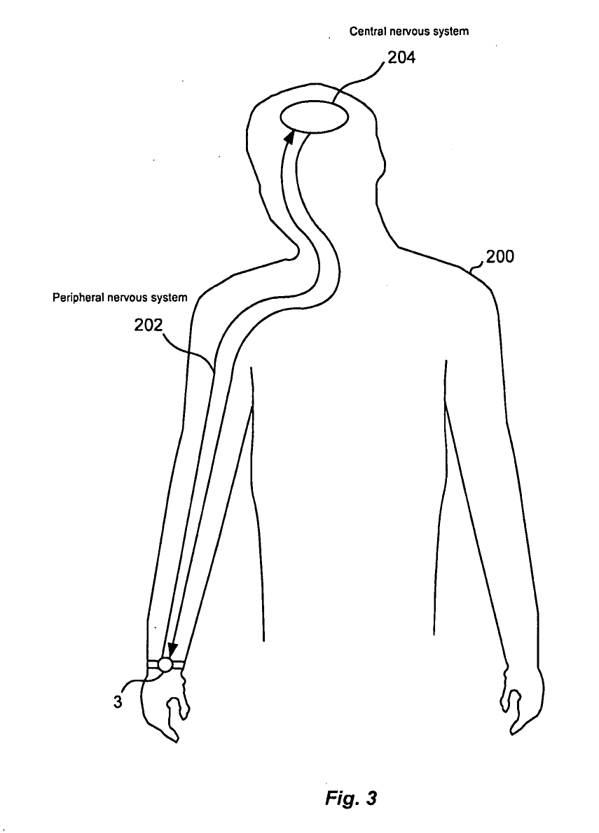

In one embodiment, the non-optical excitation is meant to excite the peripheral nervous system of the user. The peripheral nervous system resides or extends outside the central nervous system to serve the limbs and organs of the user. The central nervous system may include the brain and spinal cord of the user. Unlike the central nervous system, the peripheral nervous system is not protected by bone, leaving it exposed to toxins and mechanical injuries. FIG. 3 shows an example of the peripheral nervous system and central nervous system according to one embodiment. As shown, device 3 is being worn around the wrist by a user 1. The excitation may occur in the area around the wrist by a vibration, for example. A peripheral nervous system 202 may detect the excitation. For example, a sensory system of the peripheral nervous system 202 may sense the non-optical excitation. Signals may travel through the peripheral nervous system 202 to central nervous system 204. Central nervous system 204 may respond to the excitation. For example, the response may be that the excitation is received and the user determines some kind of movement should be performed. The excitation elicits a conscious voluntary response from the user. For example, the response is not a reflex response from the user but rather a response that requires thinking. Accordingly, signals may be sent from central nervous system 204 through peripheral nervous system 202 to cause the wrist of the user to move in the desired response.

Referring back to FIG. 2, step 206 then analyzes an outcome of the non-optical excitation based on the response and the excitation that was induced. For example, the outcome could analyze the physiological effects of the response, such as the quality of movement performed, if the movement was performed at all, average response time, etc. The physiological outcome may be any physical type of outcome that is analyzed based on the response. In one embodiment, device 3 and/or a remote device coupled to device 3 may analyze the physiological outcome. The analysis of the physiological outcome may be a pass/fail analysis. Also, the analysis may also measure the quality of the response. For example, how accurately did the user move his/her wrist in the desired pattern.

In step 208, an action may be performed in response to the analysis. For example, the action may be to store the result of the analysis. Also, other actions may be taken, such as alerting the user of the outcome. For example, is the user fails the test (i.e., does not respond or does not accurately respond), then an alarm may be output to the user, such as a buzzing, sound, etc. However, alarm generation is not completely necessary if data is intended for external or posterior assessment. Other actions may be to serve as feedback in a treatment modality such a neurological rehabilitation or neurological drug delivery.

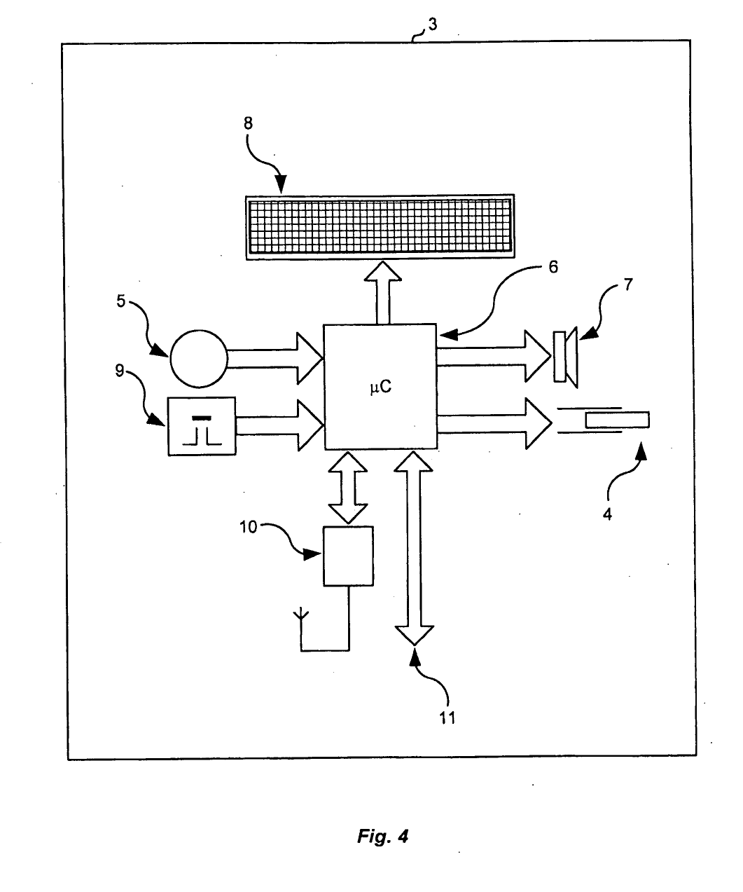

FIG. 4 shows an example of device 3 according to embodiments of the present invention. In one embodiment, device 3 is enclosed in a wristwatch case. It will be understood that components and functions of device 3 as described may be distributed to different devices. For example, a hearing aid may be used to trigger the signal and a wristwatch used to detect the response. The system can be embedded in an actual watch as a complementary function, or vice versa. It includes core elements of a digital computer or watch, that is, a microprocessor or microcontroller 6, memory and a power supply subsystem (battery and voltage regulators). It also includes movement sensors 5 as input interface and movement actuators 4 as output interface. Additional user interface elements are a display 8, a sound generation device 7 (such as beeper) and press buttons 9. Wired (e.g. USB) or wireless (e.g. Bluetooth) digital links 10,11 can also be included in order to connect the device with computers for data downloading or device programming (e.g., to select thresholds or to upgrade the neurological tests). The wireless link is used to configure the device (thresholds, interval between tests, exercise types, etc.) but also used to send data (including alarms) to a digital receiver that can be linked to a supervisor (e.g. medial doctor or family members) through a telemetric network.

In one embodiment, the movement actuators 4 are electromagnetic transducers (based on solenoids, that is, equivalent to common speakers). Piezoelectric actuators are another option and may be more efficient in terms of energy but may require higher voltages that may increase the overall complexity of the design. The use of electrical or mechanical stimulation on skin (i.e. non-optical signaling) for machine-human communication can be used.

In one embodiment, a movement sensor 5 is a micro-machined gyroscope. It is able to detect clockwise or anti-clockwise wrist turn movements and how fast these movements are produced. The response detected may be measured by the movement and the speed of the movement.

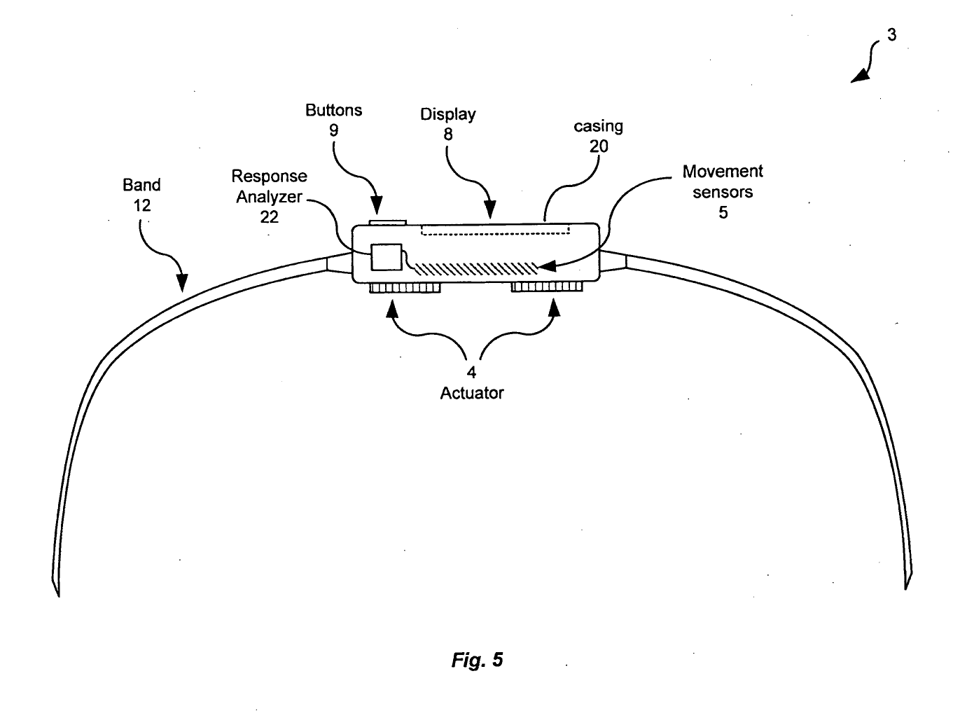

FIG. 5 shows another example of device 3 according to one embodiment. Device 3 may include a casing 20 and a band 12. Casing 20 may be configured to hold components of device 3. For example, buttons 9, display 8, movement sensors 5, and actuators 4 may be included in or on casing 20. It will be recognized that other components may be included in casing 20. For example, casing 20 may also include components of a watch or other device.

Band 12 may be included to allow user to wear device 3. For example, band 12 may be a band similar to a watch band to allow user to secure device 3 to his/her wrist. Although band 12 is shown, it will be understood that other attachment or wearable mechanisms may be provided, such as a necklace-like band. Also, band 12 may not be needed as a user may put casing 20 in his/her pocket. Also, device 3 could be fixed as a patch to any body surface.

Actuators 4 are configured to output a non-optical excitation. For example, actuators 4 may provide electrical or mechanical stimulation on the skin of a user. If a user is wearing device 3, actuator 4 may be touching or near the skin of the user. When movement of actuator 4 is caused, the user may detect the movement at their peripheral nervous system.

In the same way that movement or vibration is used to perform interrogations, non-painful electrical stimulation of different frequencies and amplitudes can also be employed by means of electrodes in contact with wrist skin beneath the watch or beneath the watch strap 12. Other stimulus sources can be: a) heat (generated from Joule effect or IR lamps), b) cold (generated from solid state cooling systems such as Peltier cells), c) chemical (such as drops of acid or oil). Also, combinations of these sources of stimulus can be employed.

When a user responds to the non-optical excitation, such as the central nervous system determines an appropriate response, movement sensors 5 detect the response. Movement sensors 5 may be movement or vibration actuators, but can be implemented by other means such as vibrating motors. Other sensors such as accelerometers, inertial switches or magnetic field sensors can be employed to detect movements. Additionally, articulation movements can be detected by other means such as strain sensors. For example, when a user moves his/her wrist in a pre-determined pattern, movement sensors 5 detect the pattern.

A response analyzer 22 is configured to analyze the response provided by the user. Although response analyzer 22 is shown as being part of device 3, it will be understood that it may be part of a remote device. Response analyzer 22 may determine if the response received is appropriate. For example, the response may be that the user did not respond at all to the excitation. Also, another response may be that the user attempted to user his/her wrist in the desired pattern. Response analyzer 22 may determine that the user attempted the movement and/or may measure the quality of the movement.

Buttons 9 and display 8 may not be used in the non-optical test. However, button 9 may be used if the user wants to activate or disable the device. Also, buttons 9 and display 8 may be used to confirm a failure of a test. For example, before triggering an alarm, a user may be asked to answer questions displayed on display 8 using buttons 9. Also, a simple question of “Please confirm you are ok?” may be asked before triggering an alarm. Also, buttons9 and display 8 may be used in setting up the parameters for the test. It should be noted that non-optical methods may be used to ask the same questions that buttons 9 and display 8 are used for, such as a user may turn his/her wrist in response to a non-optical excitation meant to ask if he/she is ok.

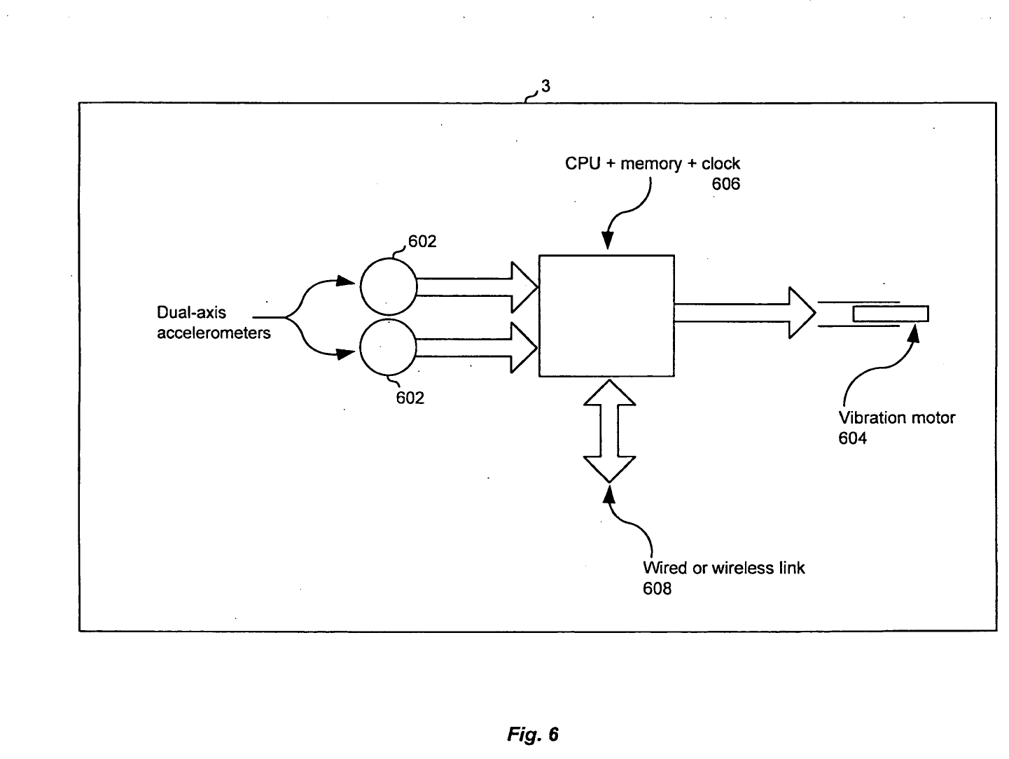

FIG. 6 shows yet another example of a neurological assessment device 3 according to one embodiment of the present invention. In one embodiment, device 3 fits into a small plastic enclosure (e.g., substantially 55 mm×35 mm×15 mm) that is fastened to the user's wrist by means of a strap. In this case, user movement is monitored with two dual-axis acceleration sensors 602 instead of using a gyroscope. Furthermore, the movement actuator consists of a vibration motor 604 instead of two electromagnetic transducers.

A CPU 606 executes simple reaction time tests at random intervals. In one example, the system interrogates the user by making the motor vibrate shortly once or twice (random selection). Then, the user must respond by turning his wrist as a soon as possible but only in the case that the motor vibrates twice, otherwise, an error is annotated. The result of each interrogation (error and time) is stored in non-volatile memory for posterior downloading through a wired or wireless link 608 into a computer for analysis.

Some examples of non-attention demanding tests are now provided. These tests can be preceded by a warning signal such as vibration or a tone. The same signal can be employed to identify the exercise that must be performed (here this signal is defined as question).

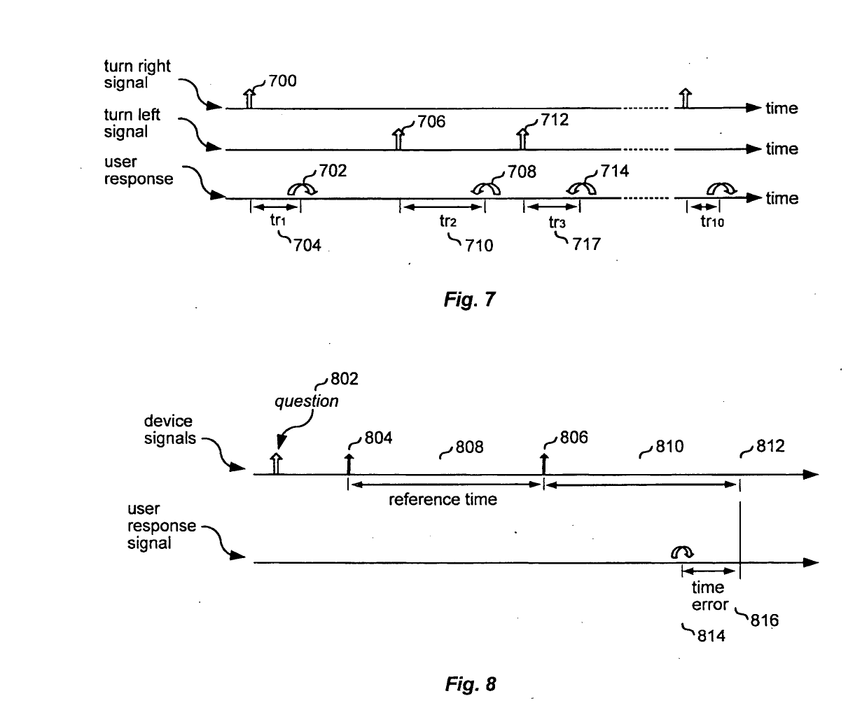

FIG. 7 shows an example of a “Reaction time test.” Randomly, in sequence, the device excites the left movement actuator or the right movement actuator and the user must turn his/her wrist in the according directions as fast as possible. Average time response (tr) is assessed to obtain the score. Accuracy can be also included in the score formula.

At 700, device 3 may output a non-optical excitation that indicates a user should perform a right-hand turn signal. The excitation may be a pattern of vibrations. For example, one vibration may be output to indicate a right-hand turn is desired.

At 702, a user response may be received. The response may be to turn device 3 in the right-hand direction. Device 3 may measure the response time (tr₁) at 704. This is the time between when the excitation is output to when the response is received.

At 706, a left-hand turn signal may be output as a second non-optical excitation. At 708, the response is received in which the user may respond with a left-hand turn movement. For example, the user may turn his/her wrist to the left-hand side. A response time 710 (tr₂) is then recorded between the time when the excitation was output and the response is received.

At 712, another turn-left signal excitation is output. At 714, the response is received and the response time TR₃ is recorded at 716. This process may continue with different non-optical signals for turning right or turning left being output. An average response time (TR) may be determined for analysis. For example, if average response time is increasing, then a possible problem may be determined. For example, a user may be experiencing problems in detecting the excitation.

A “Simple memory exercise.” may also be used. After the question corresponding to this exercise has been formulated by the device, the user must answer as soon as possible by turning his or her wrist in the opposite direction than he or she turned it in the previous simple memory exercise. Time response and accuracy are then assessed. Optionally the user can be informed of his or her errors.

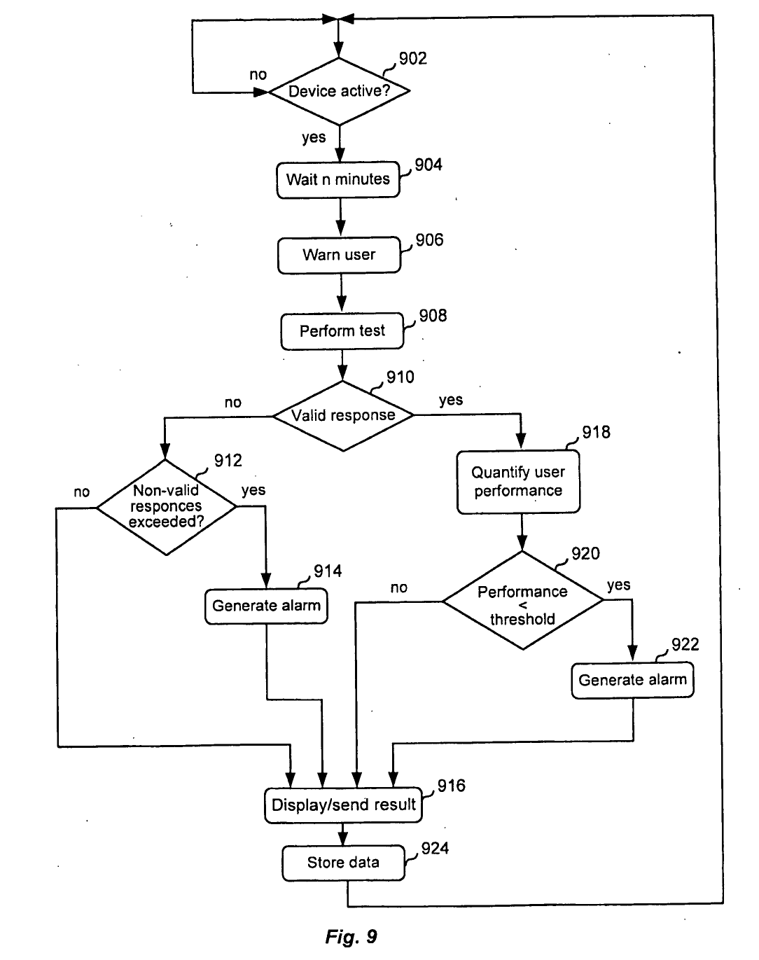

FIG. 8 shows an example of a “Pace rate estimation.” test. After the question corresponding to this exercise has been formulated, two additional signals separated by a random interval in the range of seconds are generated by the device. The user must turn his or her wrist as close as possible to the time point in which the “third” signal would be generated. Time error (in milliseconds) is assessed to obtain the score.

At 802, a question is output. At 804 and 806, two excitations are separated by a reference time 808. The user must then estimate a second reference time 810 in which a third signal 812 should be generated. At 814, the user response is received. A time error 816 may be measured to analyze the physiological outcome. For example, the error as to how close the user's response is to the answer at 812 may be measured.

A “Sequence” test may also be used. The user must follow a learned cyclic sequence of wrist turns combinations such as: 1) left-right-right, 2) right-right-right, 3) right-left-right, 4) left-left-left. After the question corresponding to this exercise has been formulated, the user must respond the combination that follows that answered in the previous exercise (since it is a cyclic sequence, the “5th” combination is “1st”). In case of error, the sequence is restarted.

A “Sequence with jumps” test may further be used. This test is the same as “sequence” exercise but in this case a random number (n) of signals are generated by the device after the question (typically from 1 to 5). User must count those signals and jump n positions in the sequence with respect to the previous answered combination. In the case of an error, the sequence is restarted.

In order to grade neurological performance and to be able to determine whether or not an alarm signal must be triggered, the device computes a “neurological performance score.” That is, in the case that the index at a time is greater than a predefined “neurological threshold,” the alarm is triggered.

Each kind of neurological exercise has its own associated assessment formula. However, in general terms, the average time response in milliseconds is added to the error rate multiplied by a scalar. Thus, for each exercise, a score expressed in equivalent time units is obtained. Then, the scores from different exercises, or from consecutive exercise series, can be averaged to obtain a “neurological performance score.”

The “neurological threshold” is not necessarily fixed. It can be adjusted for each person during the first weeks of training under controlled conditions or it can be computed by the device from a long-term average of “neurological performance scores.” Furthermore, it can be slightly scaled by the device in order to account for possible circadian rhythmic effects on neurological performance.

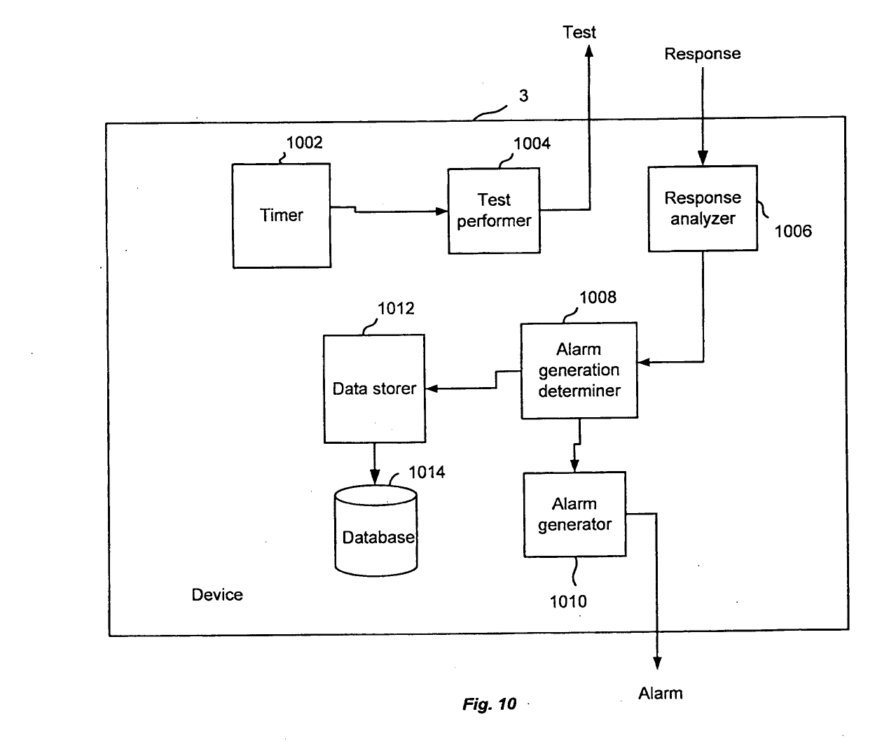

FIG. 9 depicts a flowchart 900 showing a method according to one embodiment of the present invention. The method may be executed by the microprocessor for device 3. The device can be inactive at some hours of the day or when the absence of activity (sensed by the movement sensor) indicates that the user is sleeping or is not wearing the device. When active, the device executes a loop that may perform neurological tests each n minutes (typically 1<n<30). The user can ignore the signals for a test or perform abnormal movements to indicate that he does not want to be disturbed at that moment. However, if the user rejects to answer a certain number of consecutive tests, the alarm signal is triggered. In the case that the user performs valid responses, the “neurological performance score” is computed as explained before. Then, it is averaged with some previous “neurological performance scores” and compared to the “neurological threshold”. Depending on the comparison result, the alarm signal is triggered or not.

In step 902, device 3 determines if the device should be active or not. For example, device 3 can be inactive at some hours of the day or when absence of activity (sensed by the movement sensor) indicates that the user is sleeping or not wearing the device.

When active, device 3 executes a loop that may perform neurological tests every n minutes (typically 1< and <30). For example, in step 904, device 3 waits n minutes. At 906, device 3 warns the user. For example, the warning may signal that a test is about to begin. The user can ignore the signals for the test or perform abnormal movements or a pattern that indicates that the user does not want to be disturbed at the moment.

At 908, device 3 performs the test. For example, a non-optical excitation may be output. If a non-valid response is received, device 3 may allow a user a certain number of non-valid responses. A non-valid response may be if a user does not perform the response that is required by the question, the user rejects the test, or does not perform any movement at all. In step 912, it is determined if the number of non-valid responses has been exceeded. If the number has been exceeded, at step 914 an alarm may be generated. The alarm may be to alert the user of a failure of the test. Also, an alarm signal may be sent to a remote device. For example, a customer service center may be contacted such that they can take appropriate actions, such as to call the user to check if they are ok, or to call emergency services. Optionally, the device can perform a battery of “Attention Demanding Tests” before triggering the alarm signal. For example, the user may be asked to select a button on display 8 or to perform some other test with display 8 before an alarm is triggered. If the number of non-valid responses has not been exceeded, the process proceeds to display/send the result at 916.

Referring back to step 910, if a valid response is received, at 918, device 3 may quantify the user performance. The performance may be quantified by quality. Also, the quantification may be whether or not a response is received.

At 920, device 3 determines if the performance is less than a threshold. For example, a neurological performance score may be computed. The score may include the average response time or deviation from a standard or an expected result. If the performance is less than a threshold or violates a threshold, at step 922, device 3 may generate an alarm. If the performance is not less than a threshold, then the process proceeds to display/send the result at 916. For example, the result may be displayed on device 3. Also, the result may be stored or sent to a remote device. At 924, the data may be stored for the test. For example, data may be stored in device 3 or in a remote device.

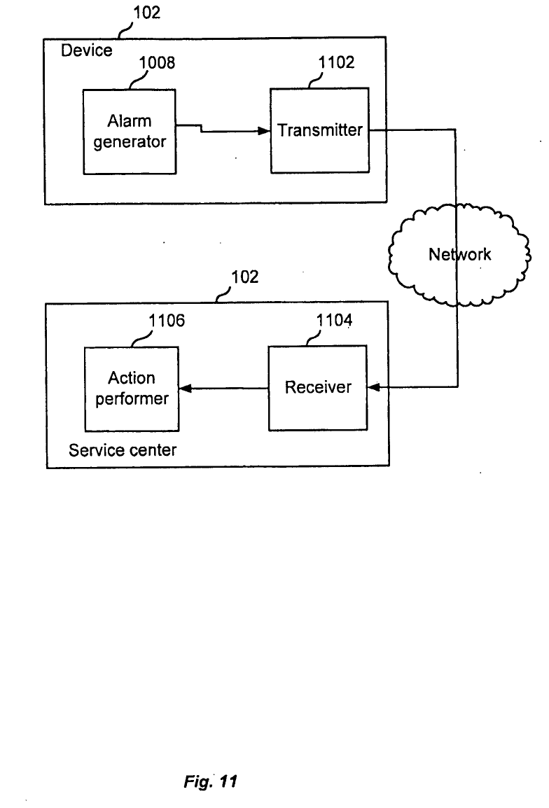

FIG. 10 shows an embodiment of a neurological assessment device. As shown, a timer 1002 and a test performer 1004 are provided. Timer 1002 is configured to determine when a test should be performed. For example, test may be performed at certain intervals.

When the time to perform a test is determined, test performer 1004 performs the test. The test may be any of the ones described.

A response analyzer 1006 determines a response received from a user. For example, response analyzer 1006 may analyze information from a gyroscope or accelerometer. The response information is then sent to an alarm generation determiner 1008. It then determines if an alarm should be generated. For example, a threshold may be used to determine if the response from the user is valid or not. Also, if the user fails the test, the user may be allowed a certain number of failures before an alarm is generated.

If alarm generation determiner 1008 determines an alarm should not be generated, a data storer 1012 may store the results for later analysis. For example, the number of consecutive failures may be stored such that it can be determined if an alarm should be later generated.

If alarm generation determiner 1008 determines an alarm should be generated, an alarm generator 1010 generates an alarm. The alarm may be generated in many ways. For example, the alarm may be generated on the device and/or sent to another system for processing. For example, FIG. 11 shows a system for processing an alarm according to one embodiment of the present invention. As shown, a transmitter 1102 may transmit an alarm to a service center. The alarm may be sent through any media, such as wirelessly, the Internet, a wired network, etc.

Receiver 1104 receives the alarm and may determine an action to take. Action perfumer 1106 is configured to perform an action. The action may be alerting an operator that an alarm has been triggered. The operator may then take some actions, such as contacting the user or contacting emergency services.

Advantages:

Previous neurological assessment systems are too obtrusive for continuous normal life monitoring. In the best of the cases, the test battery is embedded in a standard hand-held computer, such as a Personal Digital Assistant (PDA), that fits in the pocket. That implies that the user must stop whatever activity he or she is doing in order to watch a display and to press buttons (or a pressure sensitive display). Whereas in the embodiments of the present invention, the tests may be performed without requiring the user to look at a specific place and the required hand movements will be very subtle. That is, particular embodiments are suitable for continuous monitoring while performing normal life activities or while performing potentially dangerous activities. This, in turn, allows much more control through a larger number of tests and enables some applications of neurological assessment such as hypoglycemia detection.

### Applications

The Continuous Nervous System Neurological Performance Assessment Device 3 is a wearable device with the ability to induce non-optical excitation at various locations on the body through a configurable computer program. Device 3 utilizes transducers to generate non-optical stimulus then detects response in the form of specific user movements. The neurological results, mainly related to response reaction time and accuracy, can be stored in memory or sent through wired or wireless links to external examiners such as physicians or relatives or used as feedback in a neural control system, for instance for rehabilitation or drug delivery. Optionally, the device can compare the neurological results with some pre-stored thresholds and decide whether or not to trigger an alarm or to generate a feedback response.

Here described are example application fields for a wearable system for neurological assessment that do not excessively disturb most normal life activities.

### Hypoglycemia Detector

A critical drawback of insulin treatment in diabetic patients is the occurrence of hypoglycemic episodes (abnormally low blood glucose levels). Such conditions, and more specifically their consequences on the central nervous system, can seriously compromise the neurological aptitudes of the patients and can lead to seizures, stupor and coma.

With appropriate education, a lot of patients are able to detect in themselves the first symptoms of hypoglycemia and to perform glucose management. Unfortunately, some patients experience hypoglycemic unawareness. That is, those patients are not able to recognize the first symptoms and when the cognition becomes too compromised they are unable to treat themselves.

Ideally, a continuous non-invasive blood glucose monitoring device 3 would be the solution for these patients. There may be a relationship between some physiological parameters, or observable signs, and hypoglycemia. Among these are: perspiration, skin temperature, electro-cardiogram signals (ECG), electro-encephalogram signals (EEG), tremor and progressive decrease of neurological performance. Embodiments of the present invention may be used to detect the first symptoms of hypoglycemia.

In the case of hypoglycemia detection, the information from neurological assessment can be combined with information provided by other sensors (such as skin electrical conductivity) in order to implement new indexes and improve the reliability. In the case of hypoglycemia the device could be connected as real time feedback to an insulin delivery pump to deliver the insulin in need.

### Dementia Monitor

One application of device 3 is the diagnosis and continuous assessment of progressive decline in neurological function due to damage or disease in the brain beyond that which might be expected from normal aging. Such a condition is known as dementia and it affects nearly 18 million people worldwide, majority of cases being Alzheimer's disease. In some situations, sudden neurological declines may have immediately remediable causes such as medication side effects. Early recognition of neurological impairment also provides an opportunity to take advantage of the drugs available for the treatment of early stage dementia.

Aging is strongly related with a decline in neurological abilities. A significant percentage of the elderly suffer from dementia (defined as progressive decline in neurological function due to damage or disease in the brain beyond that which might be expected from normal aging). Some affected neurological areas are memory, language, attention, and problem solving. A particular type of dementia is Alzheimer's disease, which is characterized in its first stages by memory loss.

In individuals that are at risk of losing life quality due to dementia, it is important to identify the declines in neurological function indicative of imminent functional impairment. In some cases, sudden neural declines may have immediately remediable causes such as medication side effects. Moreover, early recognition of neural impairment provides an opportunity to take advantage of the newer drugs available for the early treatment of dementia.

Therefore, a wearable device 3 able to perform neural assessment would be highly valuable for patient follow-up and diagnostics. Furthermore, a wearable neural device 3 could also have some therapeutic value for dementia patients. It might be possible that continuous intellectual challenges may improve the condition of these patients or at least to minimize or delay the symptoms that they experience.

It is noted that for Alzheimer's disease patients (or with any other kind of dementia) device 3 may not only be a diagnostic instrument but also a therapeutic tool. There is sufficient epidemiological evidence to expect that continuous intellectual challenge provided by the system may improve the condition of these patients.

### Neurotoxicity Detection

Side-effects of medicines may include neural disorders and, in the worst cases, neural tissue damages. A device to continuously monitor the neural ability of patients recently prescribed these drugs could help detect and/or prevent the possible adverse effects of the medicine. Similarly, some environments pose potential for accidental exposure to neurotoxic agents. In fact, it is estimated that over 7 million U.S. workers are exposed full time to the 850+ potentially neurotoxic chemicals (many studies believe the problem may be even larger.) In those cases a wearable neural assessment system will be useful to detect the first symptoms as soon as possible and prevent unnecessary accidents, permanent mental damage, or potential lawsuits.

Currently, clinical studies that are preformed before releasing a drug to market include the study of the effects of the substance on the central nervous system. However, some individuals may experience more adverse effects than others. Thus, it would be valuable to perform continuous follow-up of patients at home and to help these patients to detect the possible adverse effects of the medicine.

In some specific environments, particularly in some industries, humans can be accidentally exposed to neurotoxic agents. In those cases, a wearable neurological assessment system will be useful to detect the first symptoms as soon as possible.

Under some conditions the delivery of neural medication may require symptomatic non-continuous delivery. A wearable neural assessment device may be valuable in serving in the feedback control loop for delivery of the drug.

### Driving, Piloting, Sports (Deep Sea Diving, Marathon Runs, High Altitude Mountain Climbing) Service Activities (Medical Emergency Teams, Fire Fighters, Military), and Use of Dangerous Machinery

Device 3 is able to assess higher mental functions (that is, neural assessment) could predict the above conditions before they become dangerous is useful. Fatigue and drug abuse are common causes of traffic accidents. Particular embodiments are able to assess higher mental functions (that is, neurological assessment) and predict drowsiness before it becomes dangerous. Moreover, a neurological challenging system will help the driver to keep a minimum level of alertness. In this case, however, it is clear that the system should not disturb the user. Therefore, user interfaces like buttons or displays are not acceptable.

Rehabilitation of Patients with Paralysis

Patients suffering from spinal cord damage causing paralysis could utilize device 3 as a form of physical therapy to occasionally remind them to try to move the affected area or to provide intelligent-feedback control stimulation sequences that would optimize the recovery. In this situation, the device 3 is not only intended to discern between sorts of movements and how fast they are triggered but also to assess their quality. That is, the device 3 quantifies parameters such as the movement speed, length, and uniformity. These parameters can also be compared with stored thresholds to trigger notifications. Such feedback information may help patients to improve their performance. Similarly, the principle can be applied to cases involving stroke victims to train the brain and recover lost or diminished function.

For rehabilitation applications the device 3 is not only intended to discern between sorts of movements and how fast they are triggered but also to assess their quality. That is, device 3 quantifies parameters such as the movement speed, length and uniformity. These parameters can also be compared with stored thresholds to trigger notifications (visual, acoustic or non-optical). Such feedback information will help patients to improve their performance.

Although the description has been described with respect to particular embodiments thereof, these particular embodiments are merely illustrative, and not restrictive.

Any suitable programming language can be used to implement the routines of particular embodiments including C, C++, Java, assembly language, etc. Different programming techniques can be employed such as procedural or object oriented. The routines can execute on a single processing device or multiple processors. Although the steps, operations, or computations may be presented in a specific order, this order may be changed in different particular embodiments. In some particular embodiments, multiple steps shown as sequential in this specification can be performed at the same time. The sequence of operations described herein can be interrupted, suspended, or otherwise controlled by another process, such as an operating system, kernel, etc. The routines can operate in an operating system environment or as stand-alone routines occupying all, or a substantial part, of the system processing. Functions can be performed in hardware, software, or a combination of both. Unless otherwise stated, functions may also be performed manually, in whole or in part.

In the description herein, numerous specific details are provided, such as examples of components and/or methods, to provide a thorough understanding of particular embodiments. One skilled in the relevant art will recognize, however, that a particular embodiment can be practiced without one or more of the specific details, or with other apparatus, systems, assemblies, methods, components, materials, parts, and/or the like. In other instances, well-known structures, materials, or operations are not specifically shown or described in detail to avoid obscuring aspects of particular embodiments.

A “computer-readable medium” for purposes of particular embodiments may be any medium that can contain, store, communicate, propagate, or transport the program for use by or in connection with the instruction execution system, apparatus, system, or device. The computer readable medium can be, by way of example only but not by limitation, an electronic, magnetic, optical, electromagnetic, infrared, or semiconductor system, apparatus, system, device, propagation medium, or computer memory.

Particular embodiments can be implemented in the form of control logic in software or hardware or a combination of both. The control logic, when executed by one or more processors, may be operable to perform that what is described in particular embodiments.

A “processor” or “process” includes any human, hardware and/or software system, mechanism or component that processes data, signals, or other information. A processor can include a system with a general-purpose central processing unit, multiple processing units, dedicated circuitry for achieving functionality, or other systems. Processing need not be limited to a geographic location, or have temporal limitations. For example, a processor can perform its functions in “real time,” “offline,” in a “batch mode,” etc. Portions of processing can be performed at different times and at different locations, by different (or the same) processing systems.

Reference throughout this specification to “one embodiment”, “an embodiment”, “a specific embodiment”, or “particular embodiment” means that a particular feature, structure, or characteristic described in connection with the particular embodiment is included in at least one embodiment and not necessarily in all particular embodiments. Thus, respective appearances of the phrases “in a particular embodiment”, “in an embodiment”, or “in a specific embodiment” in various places throughout this specification are not necessarily referring to the same embodiment. Furthermore, the particular features, structures, or characteristics of any specific embodiment may be combined in any suitable manner with one or more other particular embodiments. It is to be understood that other variations and modifications of the particular embodiments described and illustrated herein are possible in light of the teachings herein and are to be considered as part of the spirit and scope.

Particular embodiments may be implemented by using a programmed general purpose digital computer, by using application specific integrated circuits, programmable logic devices, field programmable gate arrays, optical, chemical, biological, quantum or nanoengineered systems, components and mechanisms may be used. In general, the functions of particular embodiments can be achieved by any means as is known in the art. Distributed, networked systems, components, and/or circuits can be used. Communication, or transfer, of data may be wired, wireless, or by any other means.

It will also be appreciated that one or more of the elements depicted in the drawings/figures can also be implemented in a more separated or integrated manner, or even removed or rendered as inoperable in certain cases, as is useful in accordance with a particular application. It is also within the spirit and scope to implement a program or code that can be stored in a machine-readable medium to permit a computer to perform any of the methods described above.

Additionally, any signal arrows in the drawings/Figures should be considered only as exemplary, and not limiting, unless otherwise specifically noted. Furthermore, the term “or” as used herein is generally intended to mean “and/or” unless otherwise indicated. Combinations of components or steps will also be considered as being noted, where terminology is foreseen as rendering the ability to separate or combine is unclear.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

The foregoing description of illustrated particular embodiments, including what is described in the Abstract, is not intended to be exhaustive or to limit the invention to the precise forms disclosed herein. While specific particular embodiments of, and examples for, the invention are described herein for illustrative purposes only, various equivalent modifications are possible within the spirit and scope, as those skilled in the relevant art will recognize and appreciate. As indicated, these modifications may be made to the present invention in light of the foregoing description of illustrated particular embodiments and are to be included within the spirit and scope.

Thus, while the present invention has been described herein with reference to particular embodiments thereof, a latitude of modification, various changes and substitutions are intended in the foregoing disclosures, and it will be appreciated that in some instances some features of particular embodiments will be employed without a corresponding use of other features without departing from the scope and spirit as set forth. Therefore, many modifications may be made to adapt a particular situation or material to the essential scope and spirit. It is intended that the invention not be limited to the particular terms used in following claims and/or to the particular embodiment disclosed as the best mode contemplated for carrying out this invention, but that the invention will include any and all particular embodiments and equivalents falling within the scope of the appended claims.

We claim:

1. A method for neurological interactive analysis, the method comprising:
inducing, at the device, a non-optical excitation for a user, wherein the device comprises a wearable device being worn by the user such that the non-optical excitation can be sensed;
detecting a response of a movement from the user;
analyzing an outcome based on the response and the non-optical excitation; and
performing an action in response to the analysis.

2. The method of claim 1, wherein the non-optical excitation comprises a minimally obtrusive neurological test that includes a haptic excitation that requires the response from the user.

3. The method of claim 1, wherein the outcome from the user comprises voluntary reaction from the user.

4. The method of claim 1, wherein performing the action comprises
triggering a notice that the outcome is not passed; and
sending the notice to a computing device configured to process failures of the test.

5. The method of claim 1, wherein performing the action comprises storing information for later analysis.

6. The method of claim 1, wherein analyzing the outcome comprises comparing the outcome to a threshold to determine if the outcome passes a test.

7. The method of claim 6, further comprising triggering an alarm based on the determination if the outcome does not pass the test.

8. The method of claim 1, wherein the non-optical excitation comprises an excitation of the user's peripheral nervous system, wherein the response comprises a response from the user's central nervous system.

9. The method of claim 1, wherein the response does not require a selection of an input on the device.

10. The method of claim 1, wherein analyzing the outcome comprises analyzing response time or a quality of the response of the movement from the user.

11. An apparatus configured to perform a neurological interactive analysis, the apparatus comprising:
one or more processors; and
logic encoded in one or more tangible media for execution by the one or more processors and when executed operable to:
induce a non-optical excitation for a user, wherein the apparatus is being worn by the user such that the non-optical excitation can be sensed;
detect a response of a movement from the user;
analyze an outcome based on the response and the non-optical excitation; and
perform an action in response to the analysis.

12. The apparatus of claim 11, wherein the non-optical excitation comprises a minimally obtrusive neurological test that includes a haptic excitation that requires the response from the user.

13. The apparatus of claim 11, wherein the outcome from the user comprises voluntary reaction from the user.

14. The apparatus of claim 11, wherein the logic operable to perform the action comprises logic further operable to:
trigger a notice that the outcome is not passed; and
send the notice to a computing device configured to process failures of the test.

15. The apparatus of claim 11, wherein the logic operable to perform the action comprises logic further operable to store information for later analysis.

16. The apparatus of claim 11, wherein the logic operable to analyze the outcome comprises logic further operable to compare the outcome to a threshold to determine if the outcome passes a test.

17. The apparatus of claim 16, wherein the logic is further operable to trigger an alarm based on the determination if the outcome does not pass the test.

18. The apparatus of claim 11, wherein the non-optical excitation comprises an excitation of the user's peripheral nervous system, wherein the response comprises a response from the user's central nervous system.

19. The apparatus of claim 11, wherein the response does not require a selection of an input on the device.

20. The apparatus of claim 11, wherein the logic operable to analyze the outcome comprises logic further operable to analyze response time or quality of the response of the movement from the user.

21. An apparatus configured to perform neurological interactive analysis, the apparatus comprising:
means for inducing, at the device, a non-optical excitation for a user, wherein the device comprises a wearable device being worn by the user such that the non-optical excitation can be sensed;
means for detecting a response of a movement from the user;
means for analyzing an outcome based on the response and the non-optical excitation; and
means for performing an action in response to the analysis.
